(19) United States
(12) Patent Application Publication
(10) Pub. No.: US 2012/0219597 A1
(43) Pub. Date: Aug. 30, 2012

(54) PHARMACEUTICAL FORMULATIONS FOR INDIBULIN

(75) Inventor: John C. Amedio, JR., Franklin, MA (US)

(73) Assignee: ZIOPHARM Oncology, Inc., Boston, MA (US)

(21) Appl. No.: 13/392,769

(22) PCT Filed: Sep. 1, 2010
(86) PCT No.: PCT/US10/47436

§ 371 Date: May 14, 2012

Related U.S. Application Data
(60) Provisional application No. 61/239,254, filed on Sep. 2, 2009.

(51) Int. Cl. A61K 31/4439; A61P 37/00; A61K 9/14; A61P 35/02; A61P 35/04; A61P 11/06; A61P 35/00

(52) U.S. Cl. 424/400; 514/339

(57) ABSTRACT

In certain embodiments, the invention relates to pharmaceutical formulations of an indolyl-3-glyoxylic acid derivative or a pharmaceutically acceptable salt thereof, such as indibulin. Methods of preparing such formulations and methods of treatment using these formulations are also described.

### CROSS REFERENCE TO RELATED APPLICATIONS

This application claims the benefit of U.S. Provisional Application Ser. No. 61/239,254 filed Sep. 2, 2009, which is incorporated by reference herein in its entirety.

### BACKGROUND OF THE INVENTION

During mitosis, a cell's DNA is replicated and then divided into two new cells. The process of separating the newly replicated chromosomes into the two forming cells involves spindle fibers constructed with microtubules, which themselves are formed by long chains of smaller protein subunits called tubulins. Spindle microtubules attach to replicated chromosomes and pull one copy to each side of the dividing cell. Without these microtubules, cell division is not possible.

Microtubules therefore are among the most important sub-cellular targets of anticancer chemotherapeutics, because they are present in all cells and are necessary for mitotic, interphase and cell maintenance functions (e.g., intracellular transport, development and maintenance of cell shape, cell motility, and possibly distribution of molecules on cell membranes). Compounds that interact with tubulin can interfere with the cell cycle by causing tubulin precipitation and sequestration, thereby interrupting many important biologic functions that depend on the microtubular class of subcellular organelles. Therefore, such compounds can potentially inhibit the proliferation of tumor cell lines derived from various organs. See, e.g., Bacher et al. (2001) Pure Appl. Chem. 73:9 1459-1464 and Rowinsky & Donehower (1991) Pharmac. Ther. 52:35-84.

A series of synthetic molecules that bind to tubulin are currently being evaluated in the preclinical or early clinical stage. Among them is the synthetic compound indibulin, having the following structure:

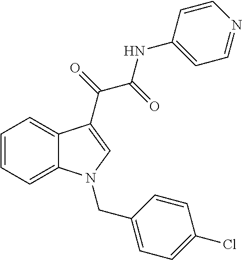

Indibulin is a synthetic small molecule tubulin inhibitor with significant antitumor activity in vitro and in vivo. It destabilizes microtubules in tumor cells, as well as in a cell-free system. The binding site of indibulin does not appear to overlap with the tubulin-binding sites of the well-characterized microtubule-destabilizing agents vincristine or colchicine. Furthermore, the molecule selectively blocks cell-cycle progression at metaphase.

The solubility of indibulin in hydrophilic solvents is poor. For example, it is practically insoluble in water, methanol, ethanol or 2-propanol. Due to these properties, the bioavailability of pure indibulin is low. A need exists for pharmaceutical formulations for indibulin which exhibit improved bioavailability.

### SUMMARY OF THE INVENTION

In certain embodiments, the invention relates to pharmaceutical formulations of an indolyl-3-glyoxylic acid derivative or a pharmaceutically acceptable salt thereof.

In certain embodiments, the indolyl-3-glyoxylic acid derivative or a pharmaceutically acceptable salt thereof has a structure of Formula (I)

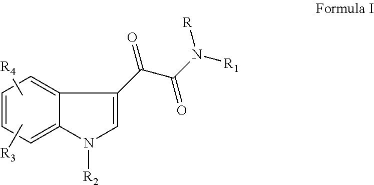

wherein

R is selected from hydrogen; (C₁-C₆)-alkyl, where the alkyl group is optionally mono- or polysubstituted with a phenyl ring which is optionally mono- or polysubstituted with halogen, (C₁-C₆)-alkyl, (C₃-C₇)-cycloalkyl, carboxyl, carboxyl esterified with C₁-C₆-alkanol, trifluoromethyl, hydroxyl, methoxy, ethoxy, benzyloxy or a benzyl group which is mono- or polysubstituted on the phenyl moiety with (C₁-C₆)-alkyl groups, halogen or trifluoromethyl; benzyloxycarbonyl; tertiary-butoxycarbonyl; and acetyl;

R₁ is selected from a phenyl ring, which is optionally mono- or polysubstituted with (C₁-C₆)-alkyl, (C₁-C₆)-alkoxy, cyano, halogen, trifluoromethyl, hydroxyl, benzyloxy, nitro, amino, (C₁-C₆)-alkylamino, (C₁⁻C₆)-alkoxycarbonylamino, carboxyl, or by carboxyl esterified with C₁-C₆-alkanol; a pyridine structure of the Formula (II)

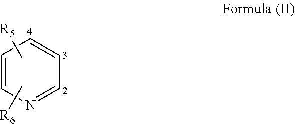

or an N-oxide thereof, where the pyridine structure is alternatively bonded to the ring carbon atoms 2, 3 or 4 and is optionally substituted with the substituents R₅ and R₆, wherein R₅ and R₆ are identical or different and are selected from (C₁-C₆)-alkyl, (C₃-C₇)-cycloalkyl, (C₁-C₆)-alkoxy, nitro, amino, hydroxyl, halogen, trifluoromethyl, ethoxycarbonylamino, and carboxyalkyloxy in which the alkyl group comprises 1-4 C atoms; 2- or 4-pyrimidinyl, wherein the 2-pyrimidinyl ring is optionally mono- or polysubstituted with a methyl group; 2-, 3-, 4- or 8-quinolyl which is optionally substituted with (C₁-C₆)-alkyl, halogen, nitro, amino or (C₁-C₆)-alkylamino; 2-, 3-, or 4-quinolylmethyl group, where the ring carbons of the pyridylmethyl radical of the quinolyl, and the quinolylmethyl are optionally substituted with (C₁-C₆)-alkyl, (C₁-C₆)-alkoxy, nitro, amino or (C₁⁻C₆)-alkoxycarbonylamino; and allylaminocarbonyl-2-methylprop-1-yl;

R₁, in the case in which R is hydrogen, methyl, benzyl, benzyloxycarbonyl, tert-butoxycarbonyl, or acetyl, is further selected from —CH₂COOH; —CH(CH₃)—COOH; —(CH₃)₂—CH—(CH₂)₂—CH-COO—; H₃C—H₂C—CH(CH₃)—CH(COOH)—; HO—H₂C—CH(COOH)—; phenyl-CH₂—CH(COOH)—; (4-imidazolyl)-CH₂—CH—(COOH)—; HN=(NH₂)—NH—(CH₂)₃—CH(COOH)—; H₂N—(CH₂)₄—CH(COOH)—; H₂N—CO—CH₂—CH—(COOH)—; and HOOC—(CH₂)₂—CH(COOH)—;

R₁, in the case in which R is hydrogen, benzyloxycarbonyl, tert-butoxycarbonyl, acetyl or benzyl, may be the acid radical of a natural or unnatural amino acid (e.g. α-glycyl, α-sarcosyl, α-seryl, α-phenylalanyl, α-histidyl, α-prolyl, α-arginyl, α-lysyl, α-asparagyl or α-glutamyl), where the amino groups of the respective amino acids may be protected or unprotected, wherein suitable protecting groups include, but are not limited to, benzyloxycarbonyl, tert-butoxycarbonyl, or acetyl, and in the case where R₁ is asparagyl or glutamyl, the second, unbonded carboxyl group is present as a free carboxyl group or in the form of an ester of a C₁-C₆-alkanol (e.g. as a methyl, ethyl or as a tert-butyl ester);

R and R₁ can further form, together with the nitrogen atom to which they are bonded, a piperazine ring of the Formula (III) or a homopiperazine ring, provided R₁ is an aminoalkylene group, in which

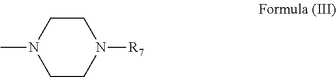

R₇ is selected from alkyl; phenyl which is optionally mono- or polysubstituted with (C₁-C₆)-alkyl, (C₁-C₆)-alkoxy, halogen, nitro, amino or by (C₁-C₆)-alkylamino; benzhydryl and bis-p-fluorobenzhydryl;

R₂ is selected from hydrogen; (C₁-C₆)-alkyl, wherein the alkyl group is optionally mono- or polysubstituted with halogen, phenyl (wherein the phenyl is optionally mono- or polysubstituted with halogen, (C₁-C₆)-alkyl, (C₃-C₇)-cycloalkyl, carboxyl, carboxyl esterified with C₁-C₆-alkanol, trifluoromethyl, hydroxyl, methoxy, ethoxy or benzyloxy), 2-quinolyl (optionally mono- or polysubstituted with halogen, (C₁-C₄)-alkyl or (C₁-C₄)-alkoxy), or 2-, 3- or 4-pyridyl (optionally mono- or polysubstituted with halogen, (C₁-C₄)-alkyl or (C₁-C₄)-alkoxy); aroyl (where the phenyl ring of the aryl moiety is optionally mono- or polysubstituted with halogen, (C₁-C₆)-alkyl, (C₃-C₇)-cycloalkyl, carboxyl, carboxyl esterified with C₁-C₆-alkanol, trifluoromethyl, hydroxyl, methoxy, ethoxy or benzyloxy);

R₃ and R₄ are identical or different and are selected from hydrogen, (C₁-C₆)-alkyl, (C₃-C₇)-cycloalkyl, (C₁-C₆)-alkanoyl, (C₁-C₆)-alkoxy, halogen, benzyloxy, nitro, amino, (C₁-C₄)-mono or dialkyl-substituted amino, (C₁-C₆)-alkoxycarbonylamino and (C₁-C₆)-alkoxycarbonylamino-(C₁-C₆)-alkyl;

Z is O or S.

In certain embodiments R₂ is selected from (C₁-C₆)-alkyl, wherein the alkyl group is optionally mono- or polysubstituted with halogen, phenyl (wherein the phenyl is optionally mono- or polysubstituted with halogen, (C₁-C₆)-alkyl, (C₃-C₇)-cycloalkyl, carboxyl, carboxyl esterified with C₁-C₆-alkanol, trifluoromethyl, hydroxyl, methoxy, ethoxy or benzyloxy), 2-quinolyl (optionally mono- or polysubstituted with halogen, (C₁-C₄)-alkyl or (C₁-C₄)-alkoxy), or 2-, 3- or 4-pyridyl (optionally mono- or polysubstituted with halogen, (C₁-C₄)-alkyl or (C₁-C₄)-alkoxy); aroyl (where the aryl moiety is optimally mono- or polysubstituted with halogen, (C₁-C₆)-alkyl, (C₃-C₇)-cycloalkyl, carboxyl, carboxyl esterified with C₁-C₆-alkanol, trifluoromethyl, hydroxyl, methoxy, ethoxy or benzyloxy).

In certain embodiments the indolyl-3-glyoxylic acid derivative is an N-substituted indole-3-glyoxylamide or a pharmaceutically acceptable salt thereof. In certain embodiments, the indolyl-3-glyoxylic acid derivative is indibulin.

One aspect of the present invention relates to solid oral dosage forms comprising particles (containing indibulin or another indolyl-3-glyoxylic acid) having a D90 particle size in the range of 250-1250 microns, an excipient, an emulsifier, a disintegrant, a diluent, and a lubricant in a weight ratio with the indibulin or other indolyl-3-glyoxylic acid of about 1:1 to 1:3, wherein any two or more of the excipient, emulsifier, disintegrant, diluent, and lubricant may be a single component. In certain embodiments, the D90 particle size is in the range of 500-1000 microns. In certain embodiments, the solid oral dosage form includes from 50 to 400 mg of indibulin or another indolyl-3-glyoxylic acid per single oral dosage unit.

In certain embodiments of the solid oral dosage form, the particles have a weight ratio of excipient, emulsifier, disintegrant, diluent, and lubricant (taken together) to indibulin or another indolyl-3-glyoxylic acid in the range of about 1:1 to 3:1, and preferably in the range of about 1.5:1 to 2:1.

In certain embodiments of the solid oral dosage form, the particles have a weight ratio of excipient to diluent in the range of about 1:3 to 1:6, and preferably in the range of about 1.4 to 1:5.

In certain embodiments of the solid oral dosage form, the particles have a weight ratio of emulsifier, diluent, and lubricant (taken together) to disintegrant in the range of about 29:1 to 40:1, and preferably in the range of about 25:1 to 35:1, preferably about 30:1.

Methods of preparing such formulations are also described. For example, in one embodiment the invention provides a process for the preparation of a solid dosage form of indibulin or another indolyl-3-glyoxylic acid, the process comprising:

a) blending indibulin or another indolyl-3-glyoxylic acid with an excipient, and emulsifier, a disintegrant and a diluent under hot melt conditions to produce a granulate;

b) blending the granulate of step a with one or more lubricants, and optionally a disintegrant, to form particles; and

c) formulating the particles of step b into a solid oral dosage form.

In certain embodiments, the method can be used to produce a solid oral dosage forms comprising particles, containing indibulin or another indolyl-3-glyoxylic acid, having a D90 particle size in the range of 250-1250 microns, an excipient, an emulsifier, a disintegrant, a diluent, and a lubricant in a weight ratio with the indibulin or other indolyl-3-glyoxylic acid of about 1:1 to 1:3, wherein any two or more of the excipient, emulsifier, disintegrant, diluent, and lubricant may be a single component. In certain embodiments, the D90 particle size is in the range of 500-1000 microns. In certain embodiments, the solid oral dosage form includes from 50 to 400 mg of indibulin or another indolyl-3-glyoxylic acid per single oral dosage unit.

In certain embodiments of the solid oral dosage form, the particles have a weight ratio of excipient, emulsifier, disintegrant, diluent, and lubricant (taken together) to indibulin or another indolyl-3-glyoxylic acid in the range of about 1:1 to 3:1, and preferably in the range of about 1.5:1 to 2:1.

In certain embodiments of the solid oral dosage form, the particles have a weight ratio of excipient to diluent in the range of about 1:3 to 1:6, and preferably in the range of about 1.4 to 1:5.

In certain embodiments of the solid oral dosage form, the particles have a weight ratio of emulsifier, diluent, and lubricant (taken together) to disintegrant in the range of about 29:1 to 40:1, and preferably in the range of about 25:1 to 35:1, preferably about 30:1.

Another aspect of the invention relates to a method of treating asthma or allergies, comprising administering a pharmaceutical formulation comprising an indolyl-3-glyoxylic acid derivative or a pharmaceutically acceptable salt thereof (such as indibulin).

Another aspect of the invention relates to a method for suppressing or inducing regression of an immunological response in a subject, comprising administering a pharmaceutical formulation comprising an indolyl-3-glyoxylic acid derivative or a pharmaceutically acceptable salt thereof (such as indibulin).

Another aspect of the invention relates to a method for treating tumors or oncoses, comprising administering a pharmaceutical formulation comprising an indolyl-3-glyoxylic acid derivative or a pharmaceutically acceptable salt thereof (such as indibulin).

Another aspect of the invention relates to a method for treating a neoplastic disease selected from leukemia, prostate carcinoma, ovarian carcinoma, epidermal carcinoma, and dunning tumor, comprising administering a pharmaceutical formulation comprising an indolyl-3-glyoxylic acid derivative or a pharmaceutically acceptable salt thereof (such as indibulin).

Another aspect of the invention relates to a method for treating antitumor agent resistant tumors, metastasizing carcinoma including development and spread of metastases, tumors sensitive to angiogenesis inhibitors or tumors that are both antitumor agent-resistant and sensitive to angiogenesis inhibitors, comprising administering a pharmaceutical formulation comprising an indolyl-3-glyoxylic acid derivative or a pharmaceutically acceptable salt thereof (such as indibulin).

Another aspect of the invention relates to a method for inhibiting multidrug-resistant tumor growth or inhibiting metastasis, comprising administering a pharmaceutical formulation comprising an indolyl-3-glyoxylic acid derivative or a pharmaceutically acceptable salt thereof (such as indibulin).

Another aspect of the invention relates to a method of treating a variety of hyperproliferative disorders, malignancies and neoplasms (e.g., solid tumors) with pharmaceutical formulations comprising an indolyl-3-glyoxylic acid derivative or a pharmaceutically acceptable salt thereof (such as indibulin). Such hyperproliferative disorders, malignancies, and neoplasms include, but are not limited to, cancers of the abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital. Other hyperproliferative disorders include, but are not limited to, hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other hyperproliferative disease, besides neoplasia, located in an organ system listed above.

Another aspect of the invention relates to a method for treating a cancer selected from cervical cancer, colon cancer, brain cancer, liver cancer, leukemia, adenoid cystic carcinoma, renal cell carcinoma, carcinoma, sarcoma, Ewing's sarcoma, leiomyosarcoma, pancreatic cancer, periampullary cancer, neuroendoplastic tumors, osteosarcoma, breast cancer, ovarian cancer, prostate cancer, vulvar cancer, glioblastoma, and lung cancer, comprising administering a pharmaceutical formulation comprising an indolyl-3-glyoxylic acid derivative or a pharmaceutically acceptable salt thereof (such as indibulin).

### DETAILED DESCRIPTION OF THE INVENTION

I. Formulations of indolyl-3-glyoxylic Acid Derivatives

In certain embodiments, the invention relates to pharmaceutical formulations, particularly an oral dosage form, for administration to a subject which include a therapeutically effective amount of one or more indolyl-3-glyoxylic acid derivatives, such as indibulin. The formulation may be in the form of a pill, tablet, capsule or powder to be administered orally. In certain embodiments, the formulation is in the form of a capsule for oral administration.

In certain embodiments, the formulation comprises an excipient, an emulsifier/solubilizer, a disintegrant, a diluent, and a lubricant in addition to, e.g., admixed with, the indolyl-3-glyoxylic acid derivatives, such as indibulin. The formulation may further comprise one or more additional diluents, disintegrants or lubricants and additional carriers.

In certain embodiments, an oral dosage form comprises from about 1 mg to about 500 mg of the indolyl-3-glyoxylic acid derivatives, such as indibulin. In certain such embodiments, the oral dosage form may comprise from about 50 mg to about 400 mg or from about 75 mg to about 300 mg of the indolyl-3-glyoxylic acid derivatives, such as indibulin. In certain embodiments, the formulation comprises about 50 mg, about 75 mg, about 100 mg, about 125 mg, about 150 mg, about 175 mg, about 200 mg, about 225 mg, about 250 mg, about 275 mg, or even about 300 mg of the indolyl-3-glyoxylic acid derivatives, such as indibulin.

In certain embodiments, the excipient may be selected from any one or more of vitamin E TPGS (D-alpha-tocopheryl polyethylene glycol 1000 succinate), Solutol (e.g., Solutol HS15), Cremophor (e.g., Cremophor RH40), and polyoxylglycerides (e.g., lauroyl or stearoyl polyoxylglycerides).

In certain preferred embodiments, the excipient is a polyethylene glycol glyceride (also known as polyoxylglycerides) composed of mono-, di- and triglycerides and mono- and diesters of polyethylene glycol (PEG), and include saturated polyglycolyzed glycerides.

In some preferred embodiments, the excipient is a Gelucire. Gelucire compositions are polyglycolized glycerides that are prepared by the alcoholysis reaction of natural oils with polyethylene glycols (PEG). They are mixtures of monoesters, diesters and/or triesters of glycerides of long chain (C12 to C18) fatty acids, and PEG (mono- and/or di-) esters of long chain (C12 to C18) fatty acids and can include free PEG. Gelucire compositions are generally described herein as fatty acid esters of glycerol and PEG esters or as polyglycolized glycerides. Gelucires are surface active in nature and disperse or solubilize in aqueous media forming micelles, microscopic globules or vesicles. They are identified by their melting point/HLB value. The melting point is expressed in degrees Celsius and the HLB (Hydrophile-Lipophile Balance) is a numerical scale extending from 0 to approximately 20. Lower HLB values denote more lipophilic and hydrophobic substances, and higher values denote more hydrophilic and lipophobic substances. The affinity of a compound for water or for oily substances is determined and its HLB value is assigned experimentally. One or a mixture of different grades of Gelucire excipient may be chosen to achieve the desired characteristics of melting point and/or HLB value. Preferred Gelucires for use in the present invention include Gelucire® 44/14, 53/10, 50/13, 42/12, and 35/10 from the Gaftefosse company, more preferably Gelucire 50/13 or Gelucire 44/14, particularly Gelucire 50/13, such as listed in CAS Registry Number: 121548-05-8.

In certain embodiments, the formulation comprises the excipient, such as Gelucire 50/13, in an amount (by weight of formulation) selected from about 5% to about 15%, about 6% to about 14%, about 7% to about 13%, about 8% to about 12%, or even about 9% to about 11%. In certain embodiments, the formulation comprises about 5%, about 6%, about 7%, about 8%, about 9%, about 10%, about 11%, about 12%, about 13%, about 14%, or even about 15% of the excipient, such as Gelucire 50/13.

In certain embodiments, the emulsifier is a mixture of fatty acid esters and/or partial esters condensed with sorbitol and/or sorbitol anhydrides. In certain embodiments, the fatty acids may be unsaturated fatty acids such as oleate and palmitate or may be saturated fatty acids such as stearate and laurate. In certain preferred embodiments, the emulsifier is a polysorbate, such as polysorbate 80, polysorbate 65, polysorbate 60, polysorbate 40 or polysorbate 20, and is preferably polysorbate 80, such as listed in CAS Registry Number 9005-65-6. In other embodiments, the emulsifier is an ester of an unmodified (i.e., non-PEG-ylated) sorbitan with fatty acids, such as Span 80, Span 65, Span 60, Span 40 or Span 20.

In certain embodiments, the emulsifier may be selected from a polysorbate (e.g., polysorbate 20, polysorbate 40, polysorbate 60, or polysorbate 80), poloxamer (e.g., poloxamer 188, poloxamer 237, poloxamer 338, or poloxamer 407), Cremophor (e.g., Cremophor EL), and a polyalkylene glycol. In certain embodiments, the formulation comprises the emulsifier, such as polysorbate 80, in an amount (by weight of formulation) selected from about 1% to about 10%, about 2% to about 9%, about 3% to about 8%, about 4% to about 7%, or even about 5% to about 6%. In certain embodiments, the formulation comprises about 1%, about 2%, about 3%, about 4%, about 5%, about 6%, about 7%, about 8%, about 9%, or even about 10% of the emulsifier, such as polysorbate 80.

The lubricant of the formulation may be selected from any one or more of talc, a silica-type lubricant such as colloidal silicon dioxide (e.g., Aerosil, Cab-O-Sil, or Syloid), starch, calcium silicate, magnesium carbonate (heavy), magnesium oxide (heavy), magnesium lauryl sulfate, sodium lauryl sulfate, calcium stearate, sodium stearyl fumarate, polyethylene glycol 4000 and/or 6000, sodium benzoate, light mineral oil, hydrogenated vegetable oils, stearic acid, and glyceryl behenate. Preferably, the lubricant includes an inert, hydrophobic lubricant.

In certain embodiments, the lubricant is a silica, such as colloidal silicon dioxide, micronized silicon dioxide or sodium aluminosilicates (such as synthetic amorphous sodium aluminosilicate). In certain embodiments, the lubricant is fumed silica such as the colloidal silicon dioxide Cab-O-Sil™.

In certain embodiments, the lubricant is a long chain fatty acid ester, such as sodium stearyl fumarate. Other exemplary fatty acid esters include sodium stearoyl lactylate, calcium stearoyl fumarate and calcium stearoyl lactate, merely to illustrate.

In certain embodiments, the lubricant is a salt of a fatty acid, preferably of a long chain (i.e., C10-C24) unsaturated fatty acid, and is preferably a metallic salt. For example, the lubricant can be magnesium stearate, or a magnesium salt of other saturated fatty acids, such as C10-C24 fatty acids. In certain embodiments, the lubricant is a metallic stearate (calcium, magnesium, and zinc).

In still other embodiments, the lubricant is a mixture of glycerides of fatty acids, such as glyceryl behenate.

In certain embodiments, the formulation may comprise a combination of lubricants, such as a silica (such as a colloidal silicon dioxide, e.g., Cab-O-Sil) and a long chain fatty acid ester (such as sodium stearyl fumarate).

In certain embodiments where the formulation is pressed into a tablet, the lubricant(s) are chosen such that the tablet has a hardness of at least 5 kg, more preferably at least 10 kg and even more preferably at least 15 kg, and/or the tablet has a tensile strength of at least 1.0 MPa, more preferably at least 1.5 MPa and even more preferably at least 2.0 MPa.

In certain embodiments, the formulation comprises the lubricant(s), such as colloidal silicon dioxide (e.g. Cab-O-Sil) and/or sodium stearyl fumarate in an amount (by weight of formulation) selected from about 0.1% to about 1%, about 0.2% to about 0.9%, about 0.3% to about 0.8%, about 0.4% to about 0.7%, or even about 0.5% to about 0.6%. In certain embodiments, the formulation comprises about 0.1%, about 0.2%, about 0.3%, about 0.4%, about 0.5%, about 0.6%, about 0.7%, about 0.8%, about 0.9%, or even about 1% of the lubricant, such as silicon dioxide (e.g., Cab-O-Sil) and/or sodium stearyl fumarate. In certain embodiments, the formulation comprises more than one lubricant, preferably two lubricants.

The diluent of the formulation may be selected from, as examples, any one or more of lactose, microcrystalline cellulose (e.g., Avicel), mannitol, calcium hydroxy-dioxido-oxo-phosphorane, dextrose, glucose, sucrose, starch and derivatives, calcium carbonate, dicalcium phosphate and magnesium carbonate.

In certain embodiments, the diluent can be a carbohydrate, such as sugar or sugar alcohols (e.g., lactose, α-lactose monohydrate, sucrose, mannitol or sorbitol) or a cellulose polymer, such as microcrystalline cellulose, silicified microcrystalline cellulose or powdered cellulose. In certain embodiments, the diluent is microcrystalline cellulose (such as Avicel PH-101).

In certain embodiments, the formulation comprises from about 25% to about 75%, about 30% to about 70%, about 35% to about 65%, about 40% to about 60%, or even about 45% to about 55% of the diluent, such as microcrystalline cellulose (e.g., Avicel PH-101). In certain embodiments, the formulation comprises about 25%, about 30%, about 35%, about 40%, about 45%, about 50%, about 55%, about 60%, about 65%, about 70%, or even about 75% of the of the diluent, such as microcrystalline cellulose (e.g., Avicel PH-101).

The disintegrant of the formulation may be selected from, for example, any one or more of starch, microcrystalline cellulose (e.g., Avicel), insoluble ion exchange resins, sodium starch glycolate (e.g., Explotab), sodium carboxymethylcellulose (e.g., croscarmellose sodium, such as Ac-Di-Sol), gums (e.g., agar, guar and xanthan), alginic acid, sodium alginate and povidone (including crospovidones, such as crospovidone type A and type B).

In certain embodiments, the disintegrant is a carboxymethyl ether of starch or a salt thereof, such as sodium starch glycolate.

In certain embodiments, the disintegrant can be a carbohydrate, such as sugar or sugar alcohols (e.g., lactose, α-lactose monohydrate, sucrose, mannitol or sorbitol) or a cellulose polymer, such as microcrystalline cellulose, silicified microcrystalline cellulose or powdered cellulose. In certain embodiments, the disintegrant is a microcrystalline cellulose, such as Avicel PH-101.

In certain embodiments, the formulation may comprise a combination of disintegrants, such as sodium starch glycolate and microcrystalline cellulose (such as Avicel PH-101).

In certain embodiments, the formulation comprises from about 0.1% to about 2%, about 0.2% to about 1.8%, about 0.4% to about 1.6%, about 0.6% to about 1.4%, or even about 0.8% to about 1.2% of the disintegrant, such as sodium starch glycolate or microcrystalline cellulose (e.g., Avicel PH-101). In certain embodiments, the formulation comprises about 0.2%, about 0.4%, about 0.6%, about 0.8%, about 1%, about 1.2%, about 1.4%, about 1.6%, about 1.8%, or even about 2% of the of the disintegrant, such as sodium starch glycolate or microcrystalline cellulose (e.g., Avicel PH-101).

In certain embodiments where the formulation is pressed into a tablet, the components, particularly the disintegrant(s), are chosen such that the resulting tablet has a dissolution rate in which one half or more of the tablet dissolves in less than 120 minutes, preferably less than 90 minutes, and even more preferably less than 60 or even 30 minutes, i.e., artificial gastric fluid without enzyme at 37° C.

The formulation may further comprise one or more additional carriers such as a binder from 3-90% and a compression filler up to 98%. The formulation may further comprise a carrier selected from a second diluent, a second disintegrant, and a second lubricant. Other pharmaceutically acceptable carriers useful for these formulations are conventional. Remington's Pharmaceutical Sciences, by E. W. Martin, Mack Publishing Co., Easton, Pa., 19th Edition (1995), describes formulations suitable for pharmaceutical delivery of the compounds herein disclosed.

In certain embodiments, the formulation comprises a component that performs the function of two or more of a lubricant, a diluent, an emulsifier, an excipient, and a disintegrant, e.g., acts as both a lubricant and a disintegrant. For example, the formulation may comprise microcrystalline cellulose as both the diluent and the disintegrant. In certain such embodiments, there may or may not be one or more additional diluents and/or disintegrants in a formulation, and/or the multi-acting component is present in an amount equal to the amounts of all of the components whose functions it performs. In certain embodiments, a single component of the formulation may act as all three of a diluent, a lubricant and a disintegrant. In certain embodiments, each of a lubricant, diluent and disintegrant are compounds that are distinct from one another.

Pharmaceutical formulations can also include one or more additional active ingredients such as antimicrobial agents, anti-inflammatory agents, anesthetics, and the like.

Pharmaceutical preparations that can be used orally include push-fit capsules made of gelatin (“gelcaps”), as well as soft, sealed capsules made of gelatin, and a plasticizer, such as glycerol or sorbitol. The push-fit capsules can contain the active ingredients in admixture with carriers. In soft capsules, the active compounds may be dissolved or suspended in suitable liquids, such as fatty oils, liquid paraffin, or liquid polyethylene glycols (PEGs). In addition, stabilizers may be added.

II. Uses for Formulations of indolyl-3-glyoxylic Acid Derivatives

Another aspect of the invention relates to a method of treating asthma or allergies, comprising administering a pharmaceutical formulation comprising an indolyl-3-glyoxylic acid derivative or a pharmaceutically acceptable salt thereof (such as indibulin).

Another aspect of the invention relates to a method for suppressing or inducing regression of an immunological response in a subject, comprising administering a pharmaceutical formulation comprising an indolyl-3-glyoxylic acid derivative or a pharmaceutically acceptable salt thereof (such as indibulin).

Another aspect of the invention relates to a method for treating tumors or oncoses, comprising administering a pharmaceutical formulation comprising an indolyl-3-glyoxylic acid derivative or a pharmaceutically acceptable salt thereof (such as indibulin).

Another aspect of the invention relates to a method for treating a neoplastic disease selected from leukemia, prostate carcinoma, ovarian carcinoma, epidermal carcinoma, and dunning tumor, comprising administering a pharmaceutical formulation comprising an indolyl-3-glyoxylic acid derivative or a pharmaceutically acceptable salt thereof (such as indibulin).

Another aspect of the invention relates to a method for treating antitumor agent resistant tumors, metastasizing carcinoma including development and spread of metastases, tumors sensitive to angiogenesis inhibitors or tumors that are both antitumor agent resistant and sensitive to angiogenesis inhibitors, comprising administering a pharmaceutical formulation comprising an indolyl-3-glyoxylic acid derivative or a pharmaceutically acceptable salt thereof (such as indibulin).

Another aspect of the invention relates to a method for inhibiting multidrug-resistant tumor growth or inhibiting metastasis, comprising administering a pharmaceutical formulation comprising an indolyl-3-glyoxylic acid derivative or a pharmaceutically acceptable salt thereof (such as indibulin).

Another aspect of the invention relates to a method of treating a variety of hyperproliferative disorders, malignancies and neoplasms (e.g., solid tumors) with pharmaceutical formulations comprising an indolyl-3-glyoxylic acid derivative or a pharmaceutically acceptable salt thereof (such as indibulin). Such hyperproliferative disorders, malignancies, and neoplasms include, but are not limited to, cancers of the abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital system. Other hyperproliferative disorders include, but are not limited to, hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other hyperproliferative disease, besides neoplasia, located in an organ system listed above.

Another aspect of the invention relates to a method for treating a cancer selected from cervical cancer, colon cancer, brain cancer, liver cancer, leukemia, adenoid cystic carcinoma, renal cell carcinoma, carcinoma, sarcoma, Ewing's sarcoma, leiomyosarcoma, pancreatic cancer, periampullary cancer, neuroendoplastic tumors, osteosarcoma, breast cancer, ovarian cancer, prostate cancer, vulvar cancer, glioblastoma, and lung cancer, comprising administering a pharmaceutical formulation comprising an indolyl-3-glyoxylic acid derivative or a pharmaceutically acceptable salt thereof (such as indibulin).

### EXEMPLIFICATION

### Example 1

### Formulations

Formulation A

Gelucire 50/13 (630 g) was combined with 315 g of polysorbate 80 in a beaker and heat to 75° C. while stiffing. Stirring was continued until all of the Gelucire 50/13 had completely melted and the mixture was homogeneous. Indibulin (2254.6 g), sodium starch glycolate (Explotab) (62.0 g), and microcrystalline cellulose (Avicel pH-101) (2829.5 g) were then added to the 25-L granulator bowl. The water re-circulator was set to 63° C. and water was re-circulates through the jacketed 25-L bowl. The bowl was then closed and the powder bed was mixed with an impeller speed of 135 rpm and a chopper speed of 1500 rpm. Mixing was continued until the powder bed temperature was at least 58 ° C. The melted Gelucire 50/13 and polysorbate 80 mixture were then pumped at a rate of 310 g per minute and after three minutes, the impeller speed was increased to 200 rpm (melt addition was complete). The granulation was massed for three minutes and then granulation was terminated (total granulation time of approximately 6 minutes). The granulation was allowed to cool in the bowl for 24 hours, or until it reached ambient temperature. Using a Comil, the granulation was milled through a 1143 μm screen at 1500 rpm and the resulting material was evaluated for particle size distribution and density. The milled product was then added to a 17-L stainless steel blending container. Sodium starch glycolate (62.0 g), colloidal silicon dioxide (Cab-o-sil) (31.0 g), and sodium stearyl fumarate (PRUV) (31.0 g) were then added to the blending container and the contents of the container were mixed for 10 minutes with a turbula blender at approximately 46 rpm until uniformity was achieved. Size 00 light blue gelatin coni-snap capsules were then filled using a Profil apparatus until all formulation was consumed. The capsules were then de-dusted using an automated deduster.

Formulation B

Gelucire 50/13 (630 g) was combined with 315 g of polysorbate 80 in a beaker and heat to 60° C. while stirring. Stirring was continued until all of the Gelucire 50/13 had completely melted and the mixture was homogeneous. Indibulin (2254.6 g), croscarmellose sodium (62.0 g), and microcrystalline cellulose (Avicel pH-101) (2829.5 g) were then added to the 25-L granulator bowl. The water re-circulator was set to 58° C. and water was re-circulates through the jacketed 25-L bowl. The bowl was then closed and the powder bed was mixed with an impeller speed of 135-200 rpm and a chopper speed of 1500 rpm. Mixing was continued until the powder bed temperature was at least 58° C. The melted Gelucire 50/13 and polysorbate 80 mixture were then pumped in to the bowl and mixed for six minutes with an impeller speed of 135-200 rpm. The granulation was massed for one and half additional minutes and then granulation was terminated. The granulation was allowed to cool in the bowl overnight so that a solid was formed. Using a Turbula Type T10B Shaker mixer, the granulation was milled through a 1143 μm screen at 1500 rpm and the resulting material was evaluated for particle size distribution and density. The milled product were then remilled at 44 rpm through a 1143 μm screen. Croscarmellose sodium (62.0 g), colloidal silicon dioxide (Cab-o-sil) (31.0 g), and sodium stearyl fumarate (PRUV) (31.0 g) were weighed separately and sieved, then added to the blending container and the contents of the container were mixed for 10 minutes with a turbula blender at approximately 46 rpm until uniformity was achieved. Size 00 light blue gelatin coni-snap capsules were then filled using a Profil apparatus until all formulation was consumed. The capsules were then de-dusted using an automated deduster.

| | Percent Formulation | Weight Per Capsule |
|---|---|---|
| Component | (% wt/wt) | (mg) |
| Indibulin | 36.4% | 200 |
| Gelucire 50/13 | 10.0% | 55 |
| Polysorbate 80 | 5.0% | 27.5 |
| Microcrystalline Cellulose | 45.6% | 251 |
| Croscarmellose Sodium | 1.0% | 5.5 |
| (Intra-Granular)* | | |
| Croscarmellose Sodium | 1.0% | 5.5 |
| (Extra-Granular)** | | |
| Colloidal Silicon Dioxide | 0.5% | 2.75 |
| Sodium Stearyl Fumarate | 0.5% | 2.75 |
| Total | 100.0% | 550 |

*Added prior to granulation.
**Added after granulation was complete.

### Example 2

### Dissolution Studies—Formulation B

Dissolution testing with one capsule per vessel.

| | Percent |
|---|---|
| | Dissolution |
| Time | Hot Melt |
| (minutes) | Granulation |
| 15 | 21.9% |
| 30 | 53.2% |
| 45 | 63.6% |
| 60 | 68.4% |
| 90 | 75.3% |
| 120 | 81.0% |
| 180 | 85.6% |

Dissolution testing with three capsules per vessel.

| Time | Percent Dissolution |
|---|---|
| (minutes) | Hot Melt Granulation |
| 15 | 14.8% |
| 30 | 29.7% |
| 45 | 41.1% |
| 60 | 46.5% |
| 90 | 54.1% |
| 120 | 56.4% |
| 180 | 52.6% |

### EQUIVALENTS

Those skilled in the art will recognize, or be able to ascertain using no more than routine experimentation, numerous equivalents to the compounds and methods of use thereof described herein. Such equivalents are considered to be within the scope of this invention and are covered by the following claims.

All of the above-cited references and publications are hereby incorporated by reference.

1. An oral formulation, comprising indibulin, an excipient, an emulsifier, a disintegrant, a diluent, and a lubricant, wherein any two or more of the excipient, emulsifier, disintegrant, diluent, and lubricant may be a single component.

2. An oral formulation of claim 1, wherein the excipient is selected from vitamin E TPGS, Solutol, Cremophor, and a polyoxylglyceride.

3. An oral formulation of claim 2, wherein the excipient is a polyoxylglyceride.

4. An oral formulation of claim 3, wherein the excipient is a Gelucire, such as Gelucire 50/13, 44/14, 53/10, 42/12 or 35/10.

5. An oral formulation of claim 2, wherein the excipient comprises about 5% to about 15% of the formulation.

6. An oral formulation of claim 5, wherein the excipient comprises about 10% of the formulation.

7. An oral formulation of claim 1, wherein the emulsifier is selected from a fatty acid ester of sorbitol or pegylated sorbitol or an anhydride, a poloxamer, Cremophor, and a polyalkylene glycol.

8. An oral formulation of claim 7, wherein the emulsifier is a polysorbate.

9. An oral formulation of claim 8, wherein the emulsifier is polysorbate 80.

10. An oral formulation of claim 1, wherein the emulsifier comprises about 1% to about 10% of the formulation.

11. An oral formulation of claim 10, wherein the emulsifier comprises about 5% of the formulation.

12. An oral formulation of claim 1, wherein the lubricant is selected from any one or more of a silica, a long chain fatty acid ester, a salt of a fatty acid glycerides of fatty acid(s), and glycerides of fatty acids.

13. An oral formulation of claim 1, wherein the lubricant is selected from any one or more of talc, colloidal silicon dioxide, starch, calcium silicate, magnesium carbonate (heavy), magnesium oxide (heavy), magnesium lauryl sulfate, sodium lauryl sulfate, calcium stearate, sodium stearyl fumarate, polyethylene glycol 4000 and 6000, sodium benzoate, light mineral oil, hydrogenated vegetable oils, stearic acid, and glyceryl behenate.

14. An oral formulation of claim 1, wherein the lubricant is selected from a silica (such as colloidal silicon dioxide, micronized silicon dioxide or sodium aluminosilicates) and a long chain fatty acid ester (such as sodium stearyl fumarate), and optionally is a combination of colloidal silicon dioxide and sodium stearyl fumarate.

15. An oral formulation of claim 13, wherein the colloidal silicon dioxide is Cab-O-Sil.

16. An oral formulation of claim 1, wherein the lubricant comprises about 0.1% to about 1% of the formulation.

17. An oral formulation of claim 16, wherein the formulation comprises 0.5% Cab-O-Sil and 0.5% sodium stearyl fumarate.

18. An oral formulation of claim 1, wherein the diluent is selected from any one or more of a carbohydrate, calcium carbonate, dicalcium phosphate and magnesium carbonate, and wherein the carbohydrate(s) is optionally selected from any one or more of a sugar alcohol and a cellulose polymer, and is optionally selected from any one or more of lactose, microcrystalline cellulose, mannitol, calcium hydroxyl-dioxido-oxo-phosphorane, dextrose, glucose, sucrose, starch and derivatives.

19. An oral formulation of claim 18, wherein the diluent is microcrystalline cellulose.

20. An oral formulation of claim 19, wherein the diluent is Avicel PH-101.

21. An oral formulation of claim 1, wherein the diluent comprises about 25% to about 75% of the formulation.

22. An oral formulation of claim 21, wherein the diluent comprises about 45% of the formulation.

23. An oral formulation of claim 1, wherein the disintegrant is selected from any one or more of a carbohydrate (optionally being starch or microcrystalline cellulose), insoluble ion exchange resins, a carboxymethyl ether of starch or a salt thereof, sodium carboxymethylcellulose, gums, alginic acid, sodium alginate and povidone.

24. An oral formulation of claim 23, wherein the disintegrant is selected from sodium starch glycolate and microcrystalline cellulose.

25. An oral formulation of claim 23, wherein the disintegrant is sodium starch glycolate.

26. An oral formulation of claim 23, wherein the disintegrant comprises about 0.1% to about 2% of the formulation.

27. An oral formulation of claim 26, wherein the disintegrant comprises about 1%, of the formulation.

28. A method for the treatment of asthma or allergies, comprising administering an oral formulation of claim 1.

29. A method for suppressing or inducing regression of an immunological response, comprising administering an oral formulation of claim 1.

30. A method for treating tumors or oncoses, comprising administering an oral formulation of claim 1.

31. A method for treating a neoplastic disease selected from leukemia, prostate carcinoma, ovarian carcinoma, epidermal carcinoma, and dunning tumor, comprising administering an oral formulation of claim 1.

32. A method for treating antitumor agent resistant tumors, metastasizing carcinoma including development and spread of metastases, tumors sensitive to angiogenesis inhibitors or tumors that are both antitumor agent-resistant and sensitive to angiogenesis inhibitors, comprising administering an oral formulation of claim 1.

33. A method for inhibiting multidrug-resistant tumor growth or inhibiting metastasis, comprising administering an oral formulation of claim 1.

34. A method for treating a hyperproliferative discover, malignancy of neoplasms, comprising administering an oral formulation of claim 1.

35. A method of claim 34, wherein the hyperproliferative disorder, malignancy, or neoplasm is selected from cancers of the abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital system.

36. A method of claim 34, wherein the hyperproliferative disorder, malignancy, or neoplasm is selected from hypergammaglobulinemia, lymphoproliferative disorders, paraproteinemias, purpura, sarcoidosis, Sezary Syndrome, Waldenstron's Macroglobulinemia, Gaucher's Disease, and histiocytosis.

37. A method for treating a cancer selected from cervical cancer, colon cancer, brain cancer, liver cancer, leukemia, adenoid cystic carcinoma, renal cell carcinoma, carcinoma, sarcoma, Ewing's sarcoma, leiomyosarcoma, pancreatic cancer, periampullary cancer, neuroendoplastic tumors, osteosarcoma, breast cancer, ovarian cancer, prostate cancer, vulvar cancer, glioblastoma, and lung cancer, comprising administering an oral formulation of claim 1.

38. A solid oral dosage form comprising indibulin-containing hot melt particles having a D90 particle size in the range of 250-1250 microns, an excipient, an emulsifier, a disintegrant, a diluent, and a lubricant in a weight ratio with the indibulin of about 1:1 to 1:3, wherein any two or more of the excipient, emulsifier, disintegrant, diluent, and lubricant may be a single component.

39. The solid oral dosage form of claim 38, which includes from 50 to 400 mg of indibulin per single oral dosage unit.

40. A process for the preparation of a solid dosage from of indibulin, the process comprising:
a) blending indibulin with an excipient, and emulsifier, a disintegrant and a diluent under hot melt conditions to produce a granulate;
b) blending the granulate of a step a with one or more lubricants, and optionally a disintegrant to form particles; and
c) formulating the particles of step b into a solid oral dosage form.

41. The process of claim 40, wherein the solid oral dosage form includes from 50 to 400 mg of indibulin per single oral dosage unit.

42. The process of claim 40, wherein the particles have a weight ratio of excipient, emulsifier, disintegrant, diluent, and lubricant (taken together) to indibulin in the range of about 1:1 to 3:1.

43. The process of claim 40, wherein the particles have a weight ration of excipient to diluent in the range of about 1:3 to 1:6.

44. The process of claim 40, wherein the particles have a weight ration of emulsifier, diluent, and lubricant (take together) to disintergrant in the range of about 29:1 to 40:1.
